(19) United States
(12) Patent Application Publication
(10) Pub. No.: US 2008/0279729 A1
(43) Pub. Date: Nov. 13, 2008

(54) Aroma porcelain wall deco

(75) Inventor: Vicki Chen, Shanghai (CN)

Correspondence Address: TROXELL LAW OFFICE PLLC, SUITE 1404, 5205 LEESBURG PIKE, Falls Church, VA, 22041, US

(73) Assignee: Top Harvest Design & Manufacture Co., Ltd.

(21) Appl. No.: 11/798,323

(22) Filed: May 11, 2007

(51) Int. Cl. A61L 9/12

(52) U.S. Cl. 422/123

(57) ABSTRACT

An aroma porcelain wall deco comprises a container having aromatic agent retained therein, and concealed inside an accommodation groove of an ornamental shield that, to be hanged onto a wall, has an assembly hole drilled thereon to communicate with the accommodation groove, permitting the opening of the container to precisely position beneath the assembly hole. An extension portion of a microelement breathable and permeable body is guided through the assembly hole, and a suction wicking cord attached to the end section of the extension portion is soaked into the aromatic agent retained inside the container. Via each fine pore of the microelement breathable and permeable body, aromatic agent absorbed by the suction wicking cord can be efficiently released to contact with the atmosphere outside and, via natural physical changes generated thereby, fragrant scents can be evenly diffused in a long-lasting and continuous manner till the aromatic agent is used up.

### BACKGROUND OF THE INVENTION

The present invention relates to an aroma porcelain wall deco, comprising a container having aromatic agent retained therein and concealed inside an ornamental shield to be hanged onto a wall, and a microelement breathable and permeable body is guided through an assembly hole of the ornamental shield and mounted thereto; whereby, via each fine pore of the microelement breathable and permeable body, aromatic agent absorbed by a suction wicking cord can be efficiently released to contact with the atmosphere outside and evenly diffused long and wide with the benefits of safety, practicality, and esthetic beauty thereby.

Conventional aroma agents applied to incense or refresh the indoor air are mainly consisted of solid-state aroma wax and press-type aroma spray wherein the solid-state aroma wax must be conveyed via the blowing of wind to contact with the atmosphere so that its fragrant scent can become airborne and diffused to the surrounding area. Otherwise, the fragrant scent will only gather at the opening of a container and, thus, fail to accurately improve the indoor air thereof. Regarding the press-type aroma spray, it must be manually operated to emit the aroma stream contacting with the atmosphere so as to generate fragrant scent thereby. However, when the fragrant odor evaporates and dies out, the aroma spray thereof must be pressed again so that the indoor air can be kept fresh and fragrant again, which is quite inconvenient in application and unable to provide fragrant scents continuously. To keep the aroma fragrances diffusing in a long-lasting manner, burning-type aromatherapy candles and essential oil burners are sometimes applied for the purpose. However, in addition to the risk of lighting by fire, these burning-type aroma diffusers can also produce dangerous carbonized odor and pose a great danger to people's health in the long run. Besides, when encountering impact from outside, the burning-type aroma diffusers can easily get turned over or broken and lead to the danger of fire, which is quite dangerous in application.

### SUMMARY OF THE PRESENT INVENTION

It is, therefore, the primary purpose of the present invention to provide an aroma porcelain wall deco, comprising a container having aromatic agent retained therein and concealed inside an ornamental shield to be hanged onto a wall, and a microelement breathable and permeable body is guided through an assembly hole of the ornamental shield and mounted thereto; whereby, via the microelement breathable and permeable body with a suction wicking cord attached thereto to absorb the aromatic agent thereby, the aromatic agent can be efficiently released to contact with the atmosphere outside and evenly diffused long and wide into the surrounding area, forming a hanging-type and mural-like aroma-diffuser design with economical space saved thereby; besides, the ornamental shield has a stylistic surface that can be flexibly changed to match to the microelement breathable and permeable body to achieve the double functions of versatile decoration effect as well as economical use of interior space thereby.

It is, therefore, the second purpose of the present invention to provide an aroma porcelain wall deco wherein the microelement breathable and permeable body having fine pores evenly and densely formed on the interior and the external surface thereon is equipped with a porous property, and can absorb the aromatic agent on a complete scale so as to increase the contact area with the atmosphere outside; whereby, via each fine porous cell of the microelement breathable and permeable body, the fragrant scent is evenly diffused into the air in a long-lasting and continuous manner till the aroma agent is used up without the inconvenience of the aforementioned conventional press-type or burning-type aroma diffusers, achieving the benefits of safety, practicality and esthetic beauty in application.

### BRIEF DESCRIPTION OF THE DRAWINGS

FIG. 1 is an exploded perspective view of the present invention.

FIG. 2 is an assembled perspective view of the present invention.

FIG. 3 is an assembled cross sectional view of the present invention.

FIG. 4 is a diagram showing the present invention in application.

### DETAILED DESCRIPTION OF THE PREFERRED EMBODIMENTS

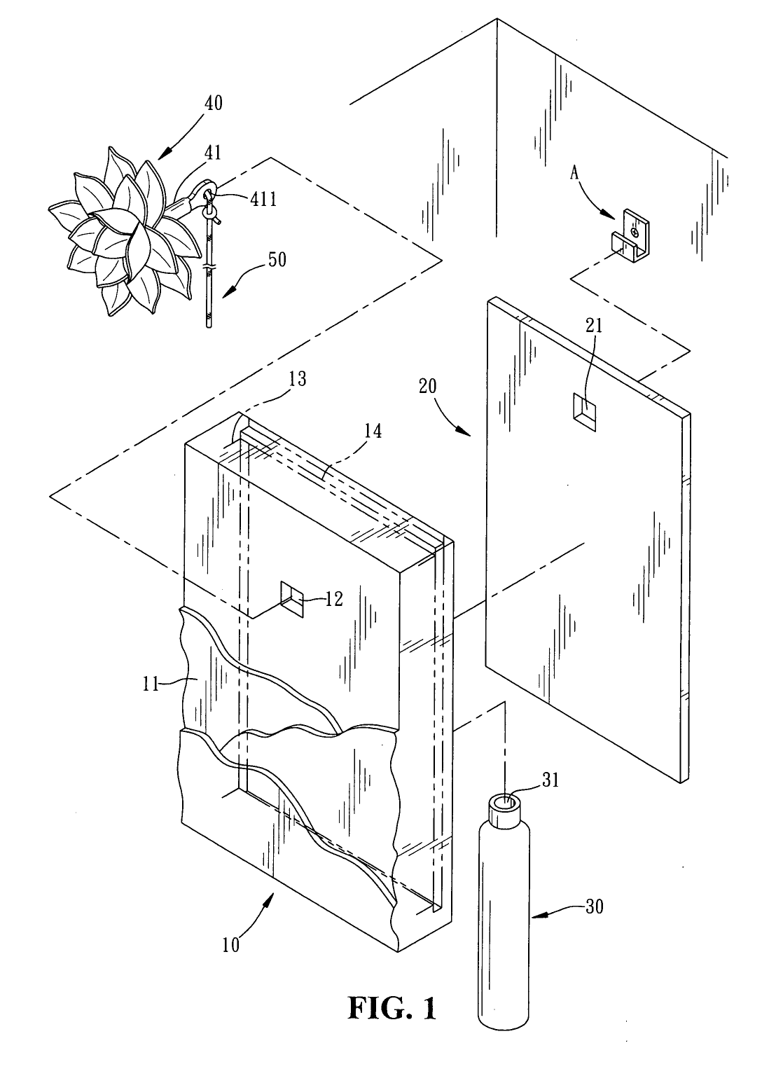

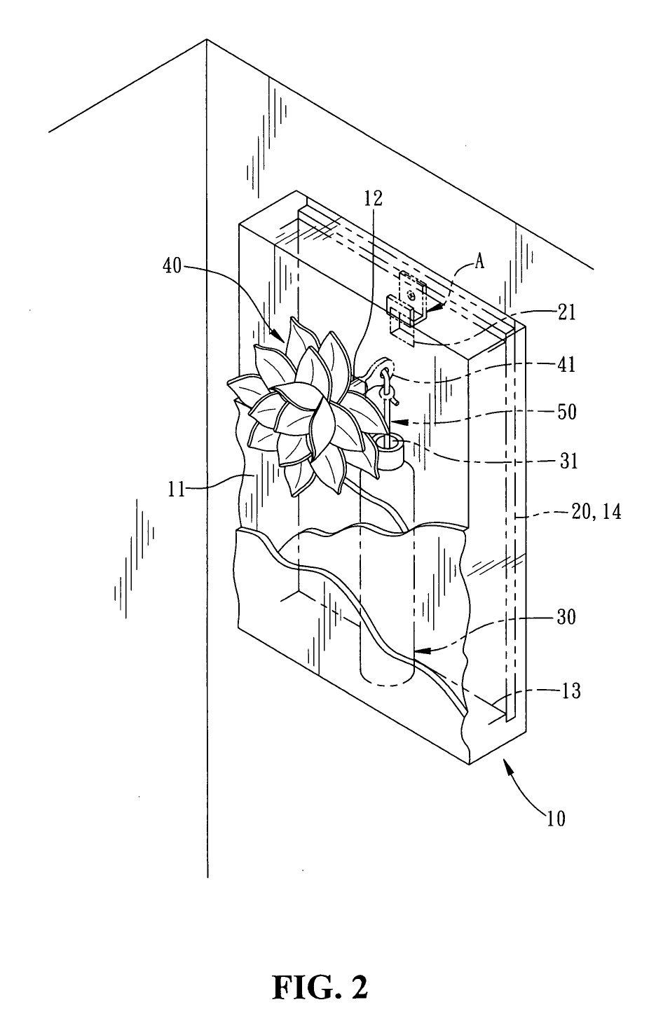

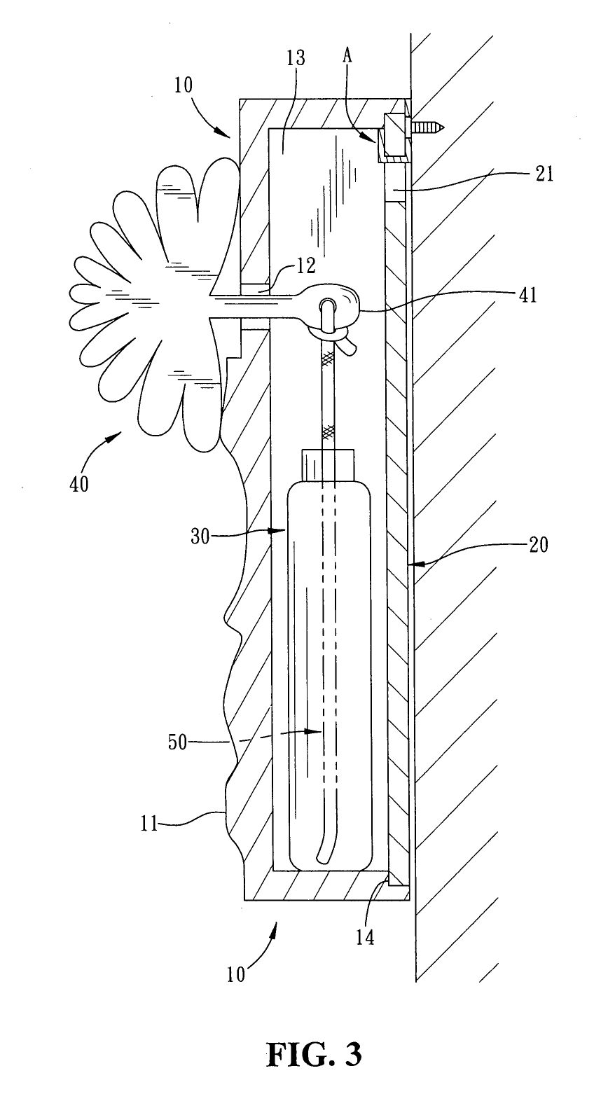

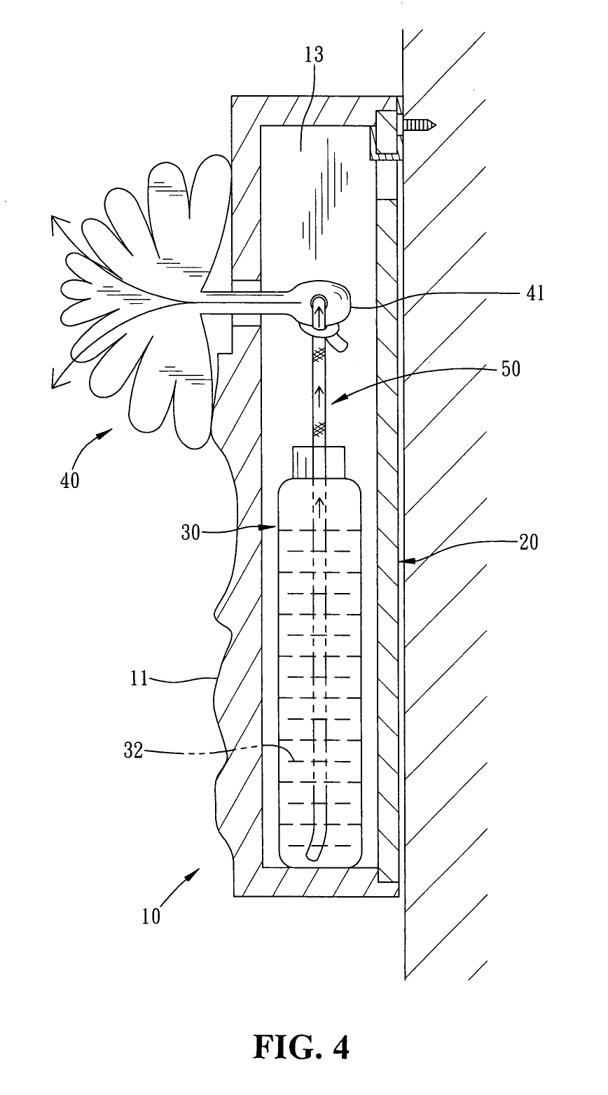

Please refer to FIG. 1 showing an exploded perspective view of the present invention (accompanied by FIGS. 2 to 4 inclusive). The present invention relates to an aroma porcelain wall deco, comprising an ornamental shield 10, a covering base 20, a container 30, a microelement breathable and permeable body 40, and a suction wicking cord 50. The ornamental shield 10 has one side embossed with three-dimensional and various-patterned stylistic surface 11 with an assembly hole 12 drilled at an appropriate position thereon, and the other side defined by an accommodation groove 13 communicating with the assembly hole 12 thereof. The accommodation groove 13 has an opening edge defined by a stepwise stop seat 14 to which the covering base 20 is precisely conjoined and engaged therewith. The stepwise stop seat 14 has a thickness identical to that of the covering base 20 so that the ornamental shield 10 can have the rear side kept in a flat and even surface thereby. The covering base 20 also has a hanging slot 21 drilled at an appropriate position thereon to be hanged onto a hook A attached to a wall surface. The container 30, having an opening 31 positioned right beneath the assembly hole 12, is provided to hold aromatic agent 32 (various type of fragrances such as essential oils) therein and be concealed inside the accommodation groove 13 of the ornamental shield 10 thereof. The microelement breathable and permeable body 40 is composed of SiO₂(35.96%), Al₂O₃(59.5%), Fe₂O₃(0.1%), TiO₂(0.04%), CaO(0.25%), MgO(0.02%), K₂O(0.82%), Na₂O(0.7%), Lg loss(2.61%), and is molded into a liquid-permeable and air-ventilating special porcelain wicking head having fine pores evenly and densely formed on the interior and the external surface thereon. The microelement breathable and permeable body 40 can be shaped into various types of objects to match to the stylistic surface 11 of the ornamental shield 10 and offer a unique-styled and mural-like decoration effect thereby. In addition, an extension portion 41 equipped with liquid-permeable and air-ventilating fine pores thereon is provided protruding at one side of the microelement breathable and permeable body 40 to be guided through the assembly hole 12 and received into the accommodation groove 13 therein. The extension portion 41 has a through hole 411 disposed at the end section thereon, to which the suction wicking cord 50 is tied and attached thereby. Thus, one end of the suction wicking cord 50 is allowed to precisely extend and soak into the aromatic agent 32 retained in the container 30 therein. Meanwhile, the suction wicking cord 50 is utilized to absorb and suck up the aromatic agent 32, and, via each fine porous cell of the microelement breathable and permeable body 40 equipped with the extension portion 41, the aromatic agent 32 is conveyed to contact with the atmosphere outside and, via natural physical changes generated thereby, fragrant scent can be evenly released and diffused outwards. Therefore, the stylistic surface 11 of the ornamental shield 10 and the microelement breathable and permeable body 40 are stylistically matched to each other to achieve a versatile decoration benefit as well as form a hanging-type mural design for more economical use of interior space. Furthermore, the microelement breathable and permeable body 40 with fine air pores evenly and densely formed on the interior and the external surface thereon is equipped with an absorbing property to suck up the aromatic agent 32 on a complete scale so as to increase the contact area with the atmosphere thereby. Via each fine porous cell of the microelement breathable and permeable body 40, the fragrant scent of the aromatic agent 32 is efficiently released and evenly diffused in the surrounding environment in a long-lasting and continuous manner till the aromatic agent 32 is used up. Thus, the present invention can avoid the inconvenience of the conventional press-type or burning-type aromatic diffusers mentioned above and provide the benefits of safety, practicality, and esthetic beauty in application.

In addition, the container 30 for retaining the aromatic agent 32 can also have the bottom side equipped with double-sided adhesive tapes or hook-and-loop fastening strips to be fixed in place onto the accommodation groove 13 of the ornamental shield 10 thereby. Thus, when the aromatic agent 32 is used up, the container 30 can be conveniently detached and dismantled there-from by consumers themselves.

The microelement breathable and permeable body 40 is composed of SiO₂(35.96%), Al₂O₃(59.5%), Fe₂O₃(0.1%), TiO₂(0.04%), CaO(0.25%), MgO(0.02%), K₂O(0.82%), Na₂O(0.7%), Lg loss(2.61%) and molded into a liquid-permeable and air-ventilating special porcelain wicking head with fine pores evenly and densely formed on the interior and the external surface thereon. High melting-point aluminum oxide is utilized as solid-phase raw material, while lower melting-point porcelain clay is used as liquid-phase material. In the first stage of the sintering process of the solid-phase material, when the powder particles reach the contact point and touch each other, the neck region at the junction between the powder particles will grow to about 20% of the grain radius, which is called the initial sintering. During this stage, the shrinkage percentage is no more than 4-5%. In the middle stage thereof, the grain boundaries will expand and the neck growth will enable the movement of the grain boundaries, resulting in the deformation of the crystal grain growth, and making the air pores at the crystal boundaries arranged into consecutive tubular shapes and form a network thereon. During this stage, the shrinkage percentage is about 5-20%, and the density also reaches 95%. Besides, the liquid-phase material thereof is added to make the clay body equipped with certain strength thereby, and the raw material with the fine air pores formed on the crystal boundaries is also utilized to provide a porous property for the absorption of essential oils and the release of the fragrant scents thereby.

What is claimed is:

1. An aroma porcelain wall deco, comprising a container with aromatic agent retained therein to be concealed inside an accommodation groove of an ornamental shield wherein the ornamental shield having a stylistic surface disposed at one side is hanged onto a wall, and an assembly hole is drilled at the stylistic surface thereon to communicate with the accommodation groove of the ornamental shield, permitting the opening of the container to precisely position beneath the assembly hole thereby; in addition, a microelement breathable and permeable body is equipped with an extension portion to be guided through the assembly hole and mounted into the accommodation groove; then, a suction wicking cord attached to the end section of the extension portion thereof is allowed to precisely extend and soak into the aromatic agent retained inside the container; the microelement breathable and permeable body is molded into a liquid-permeable and air-ventilating special porcelain wicking head having fine pores evenly and densely formed on the interior and the external surface thereon so that, via each fine porous cell of the microelement breathable and permeable body, aromatic agent absorbed by the suction wicking cord can be efficiently released to contact with the atmosphere outside and, via natural physical changes generated thereby, fragrant scent is evenly diffused outwards; therefore, the present invention can efficiently diffuse the aromatic scent in a long-lasting and continuous manner till the aromatic agent is used up without the inconvenience of the conventional press-type or burning-type aromatic diffusers, providing the benefits of safety, practicality, and esthetic beauty in application.

2. The aroma porcelain wall deco as claimed in claim 1 wherein the stylistic surface of the ornamental shield can be molded into three-dimensional and embossed various-patterned design.

3. The aroma porcelain wall deco as claimed in claim 1 wherein the accommodation groove of the ornamental shield can have a covering base reciprocally conjoined and engaged therewith.

4. The aroma porcelain wall deco as claimed in claim 3 wherein the ornamental shield has the opening edge of the accommodation groove defined by a stepwise stop seat with a thickness identical to that of the covering base so that the covering base can be precisely mounted and positioned thereto, and the ornamental shield can have the rear side kept in a flat and even surface thereby.

5. The aroma porcelain wall deco as claimed in claim 3 wherein the covering base has a hanging slot drilled at an appropriate position thereon to be hanged onto the wall surface thereby.

6. The aroma porcelain wall deco as claimed in claim 1 wherein the microelement breathable and permeable body is composed of SiO₂(35.96%), Al₂O₃(59.5%), Fe₂O₃(0.1%), TiO₂(0.04%), CaO(0.25%), MgO(0.02%), K₂O(0.82%), Na₂O(0.7%), Lg loss(2.61%), and is molded into a liquid-permeable and air-ventilating special porcelain wicking head having fine pores evenly and densely formed on the interior and the external surface thereon.

7. The aroma porcelain wall deco as claimed in claim 1 wherein the extension portion of the microelement breathable and permeable body has a through hole drilled at the end section thereon for the attachment of the suction wicking cord thereto.

8. The aroma porcelain wall deco as claimed in claim 1 wherein the container can have the bottom side equipped with double-sided adhesive tapes or hook-and-loop fastening strips to be fixed in place onto the accommodation groove of the ornamental shield thereby.
